(19) United States
(12) Patent Application Publication
(10) Pub. No.: US 2022/0392624 A1
(43) Pub. Date: Dec. 8, 2022

(54) APPARATUS AND METHOD FOR PROVIDING ARTIFICIAL INTELLIGENCE BASED VIRTUAL REALITY PSYCHOLOGICAL TEST SERVICE

(71) Applicant: PLYMIND Co.,Ltd., Seoul (KR)

(72) Inventor: Hea In JUNG, Cheonan-si (KR)

(73) Assignee: PLYMIND Co.,Ltd., Seoul (KR)

(21) Appl. No.: 17/752,791

(22) Filed: May 24, 2022

(30) Foreign Application Priority Data
May 25, 2021 (KR) 10-2021-0067043

(51) Int. Cl. G16H 40/63

(52) U.S. Cl. CPC G16H 40/63

(57) ABSTRACT

The present disclosure relates to a technique for providing an artificial intelligence based virtual reality (VR) psychological test service, and an apparatus for providing an artificial intelligence based VR psychological test service includes a test contents providing unit for providing VR contents for a psychological test to an examinee VR terminal and progressing the psychological test through a user interaction with the VR contents, a test data collecting unit for collecting test data of an examinee, and an AI analyzing unit for determining a psychological state of the examinee by analyzing the collected test data based on artificial intelligence.

### TECHNICAL FIELD

The present disclosure relates to a technique for providing an artificial intelligence based virtual reality (VR) psychological test service, and more particularly, to a method and apparatus for providing an artificial intelligence based virtual reality (VR) psychological test service which progresses a psychological test in virtual reality based on artificial intelligence (AI), collects bio-signals during the progressing of the psychological test, and accordingly, provides accurate test results.

### BACKGROUND

With the social change owing to the spread of 5G and Covid 19, the contactless era has come. The virtual reality (VR) and the augmented reality (AR) overcome the limitation of the physical space-time and are in the spotlight as core technologies that draw the contactless industry.

The representative application fields of the VR are the medical and health care fields together with the game field, the education field, and the like. With the research utilizing the VR in the rehabilitation and treatment of acrophobia and posttraumatic stress disorder (PTSD), various attempts are being made to combine the VR with the mental health treatment of modern people.

In America, there is a solution: “Virtual Vietnam” which used the VR for the first time for treating PTSD and was used to attempt to treat Vietnam war veterans at Georgia Tech in 1997. Later, Dr. Albert Rizzo, a psychiatrist at UCS in America, leads the development of the solution and utilizes the VR vigorously in treating PTSD. Eventually, the Dr. Rizzo team developed a VR solution: “Virtual Iraq” for treating PTSD in Iraq war veterans in 2005.

As such, various programs using VR contents in the mental health treatment have been developed. However, the VR application in a psychological test before a treatment is unsatisfactory yet and needs further development.

### PRIOR ART DOCUMENT

Patent document 0001) Korean registered patent No. 10-1793426 (Published on Oct. 30, 2017)

### SUMMARY

In view of the above, an embodiment of the present disclosure provides a method and apparatus for providing an artificial intelligence based virtual reality (VR) psychological test service which progresses a psychological test in virtual reality based on artificial intelligence (AI), collects bio-signals during the progressing of the psychological test, and accordingly, provides accurate test results.

An embodiment of the present disclosure also provides a method and apparatus for providing an artificial intelligence based virtual reality (VR) psychological test service which can provide a test scene through virtual reality, receive a user input response for the test using a motion of an examinee, and accordingly, provide a contactless comfortable test environment.

An embodiment of the present disclosure also provides a method and apparatus for providing an artificial intelligence based virtual reality (VR) psychological test service which can obtain an accurate test result through a big data analysis and reduce the time consumed to obtain the result in the test.

In an embodiment, an apparatus for providing an artificial intelligence based VR psychological test service includes a test contents providing unit for providing VR contents for a psychological test to an examinee VR terminal and progressing the psychological test through a user interaction with the VR contents, a test data collecting unit for collecting test data of an examinee, and an AI analyzing unit for determining a psychological state of the examinee by analyzing the collected test data based on artificial intelligence.

The test contents providing unit may progress the psychological test of the examinee in virtual reality by playing the test contents for experiencing a standardized psychological test tool according to a type of the psychological test in the virtual reality through the examinee VR terminal.

The test contents providing unit may provide a house-tree-person (HTP) drawing test by providing a test scene in front of the examinee through the examinee VR terminal, chasing a movement of a hand of the examinee by a hand glove motion sensor, and displaying a line in a movement direction of the chased hand in the test scene.

The test data collecting unit may collect biometric data together with the test data by measuring biometric signals of the examinee during the psychological test of the examinee.

The test data collecting unit may collect the test data in association with at least one biometric data of a heart rate, a body temperature, or a respiratory rate of the examinee.

The AI analyzing unit may determine a psychological state by analyzing the collected test data through a deep learning algorithm and reflecting a test item causing an emotional response through changes of the collected biometric data.

In an embodiment, the apparatus for providing an artificial intelligence based VR psychological test service may further include a test result providing unit for generating a test record sheet and a test result sheet of the examinee and providing the test record sheet and the test result sheet to the examinee or a test client.

In an embodiment, a method for providing an artificial intelligence based VR psychological test service includes progressing a psychological test of an examinee in virtual reality by playing VR contents for the psychological test, collecting test data in association with biometric data of the examinee by measuring at least one biometric data of a heart rate and a body temperature during the psychological test of the examinee, determining a psychological state of the examinee by analyzing the collected test data and the biometric data of the examinee, and generating and providing a test record sheet and a test result sheet of the examinee.

### Advantageous Effects

The present disclosure may have the following technical effects. However, this does not mean that a specific embodiment includes the entire effects or only the following effects, and accordingly, it is understood that the scope of the present disclosure is not limited thereto.

According to an apparatus for providing an artificial intelligence based virtual reality (VR) psychological test service, a psychological test is progressed in virtual reality based on artificial intelligence (AI), bio-signals during progressing the psychological test are collected, and accordingly, accurate test results can be provided.

According to an apparatus for providing an artificial intelligence based virtual reality (VR) psychological test service, a test scene is provided through virtual reality, a user input response for the test is received using a motion of an examinee, and accordingly, a contactless comfortable test environment can be provided.

According to an apparatus for providing an artificial intelligence based virtual reality (VR) psychological test service, an accurate test result can be obtained through a big data analysis, and the time consumed to obtain the result can be reduced in the test.

### BRIEF DESCRIPTION OF THE DRAWINGS

FIG. 1 is a diagram illustrating a system for providing an artificial intelligence based virtual reality (VR) psychological test service.

FIG. 2 is a block diagram illustrating a physical configuration of the apparatus for providing VR psychological test service in FIG. 1.

FIG. 3 is a block diagram illustrating a functional configuration of the apparatus for providing VR psychological test service in FIG. 1.

FIG. 4 is a flowchart illustrating an artificial intelligence based VR psychological test service providing process performed in the apparatus for providing VR psychological test service shown in FIG. 1.

FIG. 5 is an exemplary diagram illustrating an artificial intelligence based VR psychological test process according to an embodiment.

### DETAILED DESCRIPTION

The description of the present disclosure is merely an exemplary embodiment for structural and functional descriptions. This present disclosure should not be construed as limited to the embodiments set forth herein. That is, since the embodiments may have various modifications and variations, it is understood that the scope of the present disclosure includes equivalences that implement the technical concept. In addition, a specific embodiment should include all the proposed objects or effects or include only the effects, and accordingly, it should not be understood that the scope of the present disclosure is limited thereby.

Meanwhile, the terms described in the present application should be understood as follows.

The terms “first” and “second” are used to distinguish one element from another element, and the scope of the present disclosure should not be limited by these terms. For example, a first element may also be referred to as a second element, and similarly, a second element may also be referred to as a first element.

It will be understood that when an element is referred to as being “connected” to another element, it may be directly connected to the other element, or it will be understood that an element may exist therebetween. However, when an element is referred to as being “directly connected” to another element, it will be understood that there is no element therebetween. Meanwhile, other expressions describing a relation between elements, that is, “between˜”, “directly between˜”, “neighboring˜”, “adjacent to˜”, and the like should be interpreted in the same way.

A singular expression should be interpreted to include a plural expression unless it does not mean otherwise in the context, and it is understood that the term “include” or “have” indicates that a feature, a number, a step, an operation, a component, a part, or the combination thereof described in the specification is present but does not exclude a possibility of presence or addition of one or more other features, numbers, steps, operations, components, parts, or combinations thereof, in advance.

It will be understood that an identification code (e.g., a, b, c, etc.) is used for the convenience of description. The identification code is not intended to describe an order of steps, but the steps may occur in the same order as the specified order or may be performed substantially at the same time or may be performed in the opposite order.

The present disclosure may be implemented as codes readable by a computer on a recording medium readable by a computer, and the recording medium readable by a computer includes all types of recording devices in which data readable by a computer system is stored. An example of the recording medium readable by a computer includes ROM, RAM, CD-ROM, magnetic tape, floppy disk, optical data storage device, and the like. In addition, in the recording medium readable by a computer, codes readable by a computer may be distributed on a computer system connected via a network and stored and executed in a distributed manner.

Unless it is contrarily defined, all terms have the same meanings as those generally understood by a person with ordinary skill in the art. Terms which are defined in a generally used dictionary should be interpreted to have the same meaning as the meaning in the context of the related art and are not interpreted as an ideal meaning or excessively formal meanings unless clearly defined in the present application.

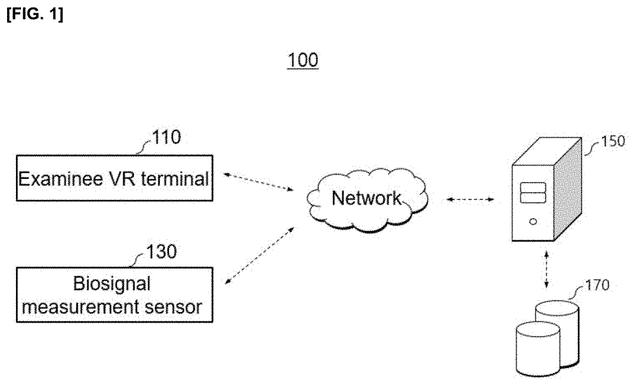

FIG. 1 is a diagram illustrating a system for providing an artificial intelligence based virtual reality (VR) psychological test service.

Referring to FIG. 1, a system for providing a VR psychological test service 100 may include an examinee VR terminal 110, a biosignal measurement sensor 130, an apparatus for providing VR psychological test service 150, and a database 170.

The examinee VR terminal 110 may correspond to a user terminal which can perform a VR psychological test service. For example, the examinee VR terminal 110 may be implemented with a head-mounted display (HMD), a VR/AR glass, and the like, representatively, but not limited thereto, and may be implemented with various types of devices which can display a VR image. The HMD is a display device which is mounted on ahead and used for a display device for implementing virtual reality or augmented reality, generally. The HMD may be connected to a 3D display technique or supported on mobile. The examinee VR terminal 110 may be connected to the apparatus for providing VR psychological test service 150 via a network and exchange data. A plurality of examinee VR terminals 110 may be simultaneously connected to the apparatus for providing VR psychological test service 150.

The biosignal measurement sensor 130 may be implemented with a device in the form of a wearable device which may be worn by an examinee. The biosignal measurement sensor 130 may measure a biosignal of the examinee in the process of progressing a psychological test of the examinee and provide the measured biosignal to the apparatus for providing VR psychological test service 150. Here, the biosignal of the examinee may include a heart rate and body temperature information, but not limited thereto, and various types of body information that causes a physical change according to a psychological state such as a pulse and a blood pressure.

The apparatus for providing VR psychological test service 150 may provide VR contents for psychological measurement to the examinee VR terminal 110 and receive test data and biometric data measuring psychology of examinee through an interaction with contents. Here, VR contents for psychological measurement may include at least one of image, sound, tactile information, and olfactory information to provide visual or verbal stimulation to examinee and may be question-type diagnostic content transmitted to examinee through synthetic voice or text selection corresponding to virtual psychologist. For example, in question-type VR content for in-depth evaluation such as basic state evaluation to recognize psychological state of examinee, developmental process evaluation to recognize attachment type and early life stress of examinee and interpersonal sensitivity evaluation through facial expression recognization may be provided to help determine psychological state of examinee.

In an embodiment, the apparatus for providing VR psychological test service 150 may expertly analyze the collected data and provide the analysis result to the examinee. The apparatus for providing VR psychological test service 150 may obtain an accurate test result by performing analysis based on artificial intelligence (AI) and provide a customized psychotherapy service by converting the test result into big data. Here, the apparatus for providing VR psychological test service 150 may implement a model capable of predicting mental health and preventing mental disease based on the big data previously constructed according to an actual psychological measurement situation.

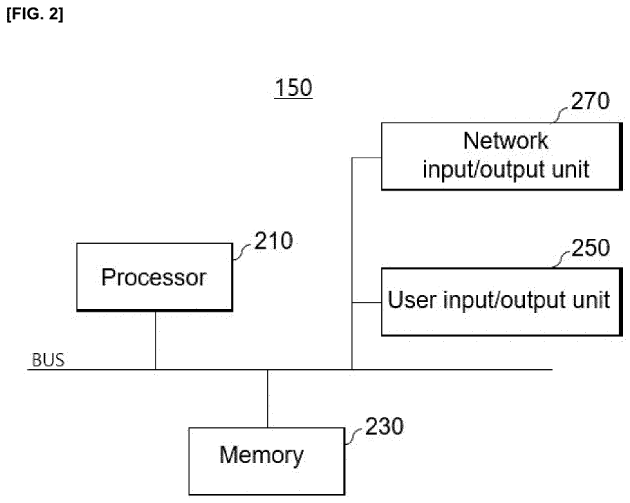

In addition, the apparatus for providing VR psychological test service 150 may be implemented by including a processor, a memory, a user input/output unit, and a network input/output unit, which will be described in more detail with reference to FIG. 2.

The database 170 may correspond to a storage device that stores various types of information required for an operation process of the apparatus for providing VR psychological test service 150. The database 170 may store examinee information, psychological test information, collected biometric information, and the psychological test result, but not be limited thereto and may store information collected or processed in various forms in the VR psychological test service process based on artificial intelligence.

In an embodiment, database 170 may be implemented independently or may be implemented by being included in the apparatus for providing VR psychological test service 150.

FIG. 2 is a block diagram illustrating a physical configuration of the apparatus for providing VR psychological test service in FIG. 1.

Referring to FIG. 2, the apparatus for providing VR psychological test service 150 may be implemented by including a processor 210, a memory 230, a user input/output unit 250, and a network input/output unit 270.

The processor 210 may execute a procedure for processing each operation of the apparatus for providing VR psychological test service 150 and may manage the memory 230 read or written throughout the process and schedule a synchronization time between the volatile memory and nonvolatile memory in the memory 230. The processor 210 may control the overall operation of the apparatus for providing VR psychological test service 150 and may be electrically connected to the memory 230, the user input/output unit 250, and the network input/output unit 270 to control data flow therebetween. The processor 210 may be implemented as a central processing unit (CPU) of the apparatus for providing VR psychological test service 150.

The memory 230 may be implemented as nonvolatile memory such as a solid state drive (SSD) or a hard disk drive (HDD) and may include an auxiliary memory device which is used to store the overall data required for the apparatus for providing VR psychological test service 150 and may also include a main memory device implemented with a volatile memory such as random access memory (RAM).

The user input/output unit 250 may include an environment for receiving a user input and an environment for outputting specific information to a user. For example, the user input/output unit 250 may include an input device including an adapter such as a touch pad, a touch screen, an image keyboard, or a pointing device and an output device including an adapter such as a monitor or a touch screen. In an embodiment, the user input/output unit 250 may correspond to a computing device connected through remote access, and in this case, the apparatus for providing VR psychological test service 150 may be performed as a server.

The network input/output unit 270 may include an environment for connecting with an external device or a system through a network and may include, for example, an adapter for communication such as LAN (Local Area Network), MAN (Metropolitan Area Network), WAN (Wide Area Network), and VAN (Value Added Network).

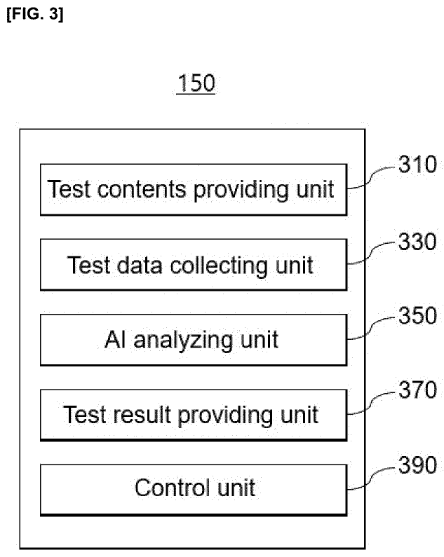

FIG. 3 is a block diagram illustrating a functional configuration of the apparatus for providing VR psychological test service in FIG. 1.

Referring to FIG. 3, the apparatus for providing VR psychological test service 150 may include a test contents providing unit 310, a test data collecting unit 330, an AI analyzing unit 350, a test result providing unit 370, and a control unit 390.

The test contents providing unit 310 may provide test contents to an examinee through the examinee VR terminal 110. Here, the test contents may correspond to contents that allow the user to experience a standardized psychological test tool in virtual reality. The test contents providing unit 310 may be requested with the psychological test from an individual, a hospital, a company, and the like, generate test contents according to the requested psychological test type, and provide the test contents to the examinee through the corresponding examinee VR terminal 110. In an embodiment, the test contents providing unit 310 may provide the test contents for performing a projective test.

The projective test is a type of propensity test for presenting ambiguous visual or verbal stimuli to the examinee and watching the process of inner perception, interpretation, and responses of the examinee, and then, evaluating an inner psychological state and a personality characteristic. The projective test include a house-tree person (HTP) picture test, a thematic apperception test (TAT), a kinetic family drawing test, a sentence completion test (SCT), and the like. The HTP test is a projective test for measuring a personality pattern, and used to analyze brain damage and general mental function, and thus, may identify the inner world of the examinee through a drawing.

In an embodiment, the test contents providing unit 310 may provide a drawing screen for the HTP test in front of the examinee through the examinee VR terminal 110. The test contents providing unit 310 may chase a gaze movement of the examinee, and when the examinee moves a gaze, may provide a drawing screen in the corresponding direction and space, that is, in front of the examinee. The examinee may take a motion of sequentially drawing a house, a tree, and a person in the drawing screen provided in the examinee VR terminal 110. The test contents providing unit 310 may receive a user input response for the drawing screen through a hand glove motion sensor. The hand glove motion sensor is a glove form interface embedded with various sensors. Here, the test contents providing unit 310 may chase the hand by the sensor attached to the hand glove worn by the examinee and display a line in a movement direction of the chased hand on the drawing screen, and thus, may input data on the drawing drawn by the examinee for the HTP test.

The test contents providing unit 310 may provide an image and a voice for presenting visual and verbal stimuli to the examinee through the examinee VR terminal 110. The test contents providing unit 310 may provide a questionnaire for a questionnaire test through the examinee VR terminal 110. The examinee may perform a psychological test through the test contents provided in the examinee VR terminal 110. The examinee may progress a psychological test according to the test contents according to a type of the psychological test in the VR test environment displayed through the examinee VR terminal 110. For example, psychological tests such as a personality type, a behavior type, depression, and anger scale may be conducted in form of question-and-answer questions from a virtual psychologist.

The test data collecting unit 330 may collect examinee biometric data in the test data and the test process. In an embodiment, the test data collecting unit 330 may collect a response of the examinee according to the test contents provided in the examinee VR terminal 110 as the test data. The test data collecting unit 330 may collect the test data by converting a drawing drawn by hand by the examinee on the VR screen for an HTP test to an image. The test data collecting unit 330 may receive and collect the answer of the examinee to a question without any change or may process an STT (Speech to Text) to collect the answer in the form of a text.

The test data collecting unit 330 may receive a biosignal of the examinee while the VR psychological test is progressed. The test data collecting unit 330 may receive and collect examinee biometric information by measuring a biometric state of the examinee through the biosignal measurement sensor 130. The examinee biometric information may include at least one of a heart rate, a body temperature, a respiratory rate, and a blood pressure of the examinee, but is not limited thereto, and may include all types of measurable physical information which is associated with a psychological state determination.

In the case that the biometric information of the examinee is measured by the biosignal measurement sensor 130, the biometric information may be transmitted to the test data collecting unit 330 in the apparatus for providing VR psychological test service 150 through a network.

The test data collecting unit 330 may collect the biometric information of the examinee while the VR psychological test is progressed and store the biometric information of the examinee for each test item to identify the test item caused by a physical change of the examinee. For example, in the case that a person undergoing a test is lying, changes in body function such as a heart rate, a blood pressure, respiratory, and sweat occur, and are due to the emotional response that the person is lying. The test data collecting unit 330 may collect the biometric information of the examinee for the test data that cause the emotional response of the examinee. The test data collecting unit 330 may generate a test record sheet by collecting the test data and the biometric data. The test record sheet may include personal information of the examinee such as a name, a gender, and an age of the examinee.

The AI analyzing unit 350 may analyze the test record sheet based on AI to determine a psychological state of the examinee and may generate the test result sheet. The psychological state of the examinee may be classified into various psychological states such as not only normal, depression, and anxiety, but also post-traumatic stress disorder, cognitive decline, and the like. In an embodiment, the AI analyzing unit 350 may determine a psychological state by extracting a characteristic from a drawing recorded in the test record sheet and analyzing the extracted feature point through artificial intelligence deep learning algorithm as an input. Here, the AI analyzing unit 350 may use the convolutional neural network (CNN) to classify the top-level item such as a house shape, a tree shape, a person shape, and the like from the drawing image recorded in the test record sheet. For example, when the drawing image is classified as a house shape, the house shape is classified into next-level items such as a window shape, a chimney shape, a thatched house shape, a tile-roofed shape, and the like, and finally, in the case of a window shape, the drawing test may be analyzed through a pre-learned model for each by classifying the items depending on the number of windows, a presence of bars in the window, and the like.

In an embodiment, the AI analyzing unit 350 may analyze the test item that causes an emotional response of the examinee based on the biometric information of the examinee and may increase the accuracy of the psychological state determination of the examinee by reflecting the test item in the psychological state determination. The AI analyzing unit 350 may analyze the question-and-answer content through AI learning model. The AI analyzing unit 350 may generate a test result sheet based on the analysis result.

The test result providing unit 370 may store the test record sheet and the test result sheet and may provide the test record sheet and the test result sheet according to a designated method when the examinee or a test client requests. In an embodiment, the test result providing unit 370 may transmit an access link to the examinee or the test client terminal in the form of an email or a message to inquire the test result.

The control unit 390 may control the overall operations of the apparatus for providing VR psychological test service 150 and may manage a control flow or a data flow among the test content providing unit 310, the test data collecting unit 330, the AI analyzing unit 350, and the test result providing unit 370.

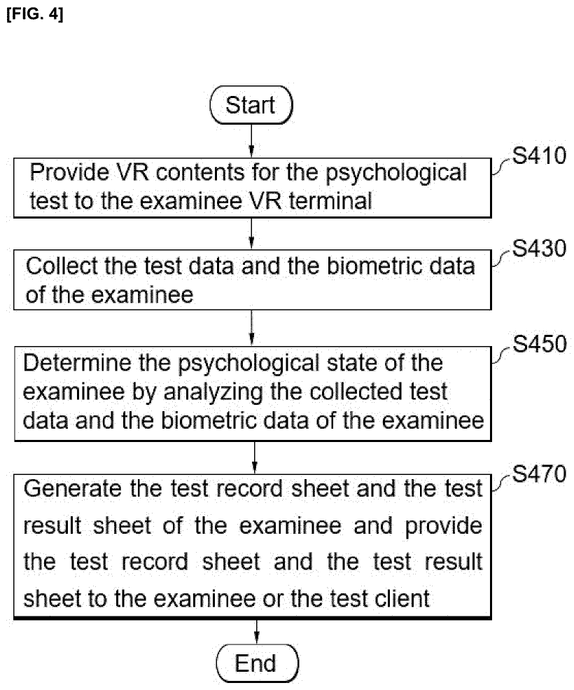

FIG. 4 is a flowchart illustrating an artificial intelligence based VR psychological test service providing process performed in the apparatus for providing VR psychological test service shown in FIG. 1.

Referring to FIG. 4, the apparatus for providing VR psychological test service 150 may provide VR contents for the psychological test through the test contents providing unit 310 to the examinee VR terminal 110 (step S410). The test contents providing unit 310 may playback the VR contents through the examinee VR terminal 110 to perform the psychological test of the examinee in the virtual reality. During the psychological test of the examinee, biosignals such as a heart rate, a body temperature, and the like of the examinee may be measured through the biosignal measurement sensor 130.

The apparatus for providing VR psychological test service 150 may collect the test data and the biometric data of the examinee through the test data collecting unit 330 (step S430). The test data collecting unit 330 may collect the test data in association with the biometric data and determine a part of the test item that causes an emotional response through a biometric change.

The apparatus for providing VR psychological test service 150 may determine the psychological state of the examinee by analyzing the test data and the biometric data of the examinee collected by the AI analyzing unit 350 (step S450). The AI analyzing unit 350 may accurately determine a psychological state of the examinee by performing the analysis of the test data and the biometric data based on the artificial intelligence.

The apparatus for providing VR psychological test service 150 may generate the test record sheet and the test result sheet of the examinee through the test result providing unit 370 and may provide the test record sheet and the test result sheet to the examinee or the test client (step S470).

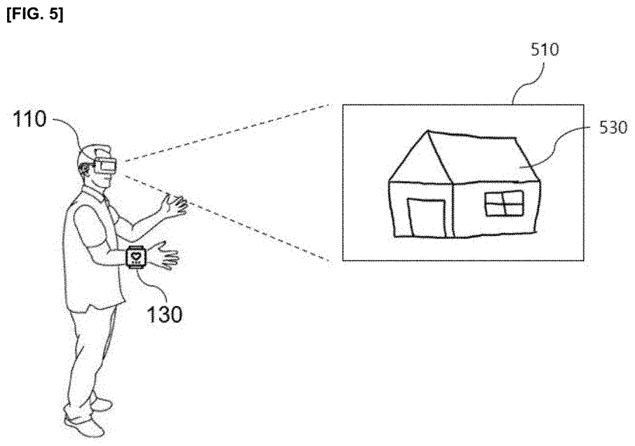

FIG. 5 is an exemplary diagram illustrating an artificial intelligence based VR psychological test process according to an embodiment.

Referring to FIG. 5, the artificial intelligence based VR psychological test service providing system 100 may receive VR contents for a psychological test through the apparatus for providing VR psychological test service 150 and may output a virtual reality image and sound for the psychological test to the examinee VR terminal 110. In this case, the examinee may perform a psychological test according to a guidance of a virtual psychologist in the virtual reality.

The apparatus for providing VR psychological test service 150 may provide VR contents according to a type of the psychological test to the examinee VR terminal 110. For example, in the case of performing an HTP test, the apparatus for providing VR psychological test service 150 may provide a test scene 510 in which the examinee may draw a house, a tree, and a person to the front of the examinee through the examinee VR terminal 110. When the examinee draws a house, a tree, and a person by the hand on the test scene 510 displayed in front of the examinee, the apparatus for providing VR psychological test service 150 may chase the movement of the hand of the examinee through the finger motion sensor and the HTP psychological test may be performed by displaying a drawing image 530 according to the movement of the hand on the test scene 510.

During the VR psychological test, the apparatus for providing VR psychological test service 150 may receive a biosignal such as a heart rate, a body temperature, and the like of examinee measured by the biometric measurement sensor 130.

According to the method and apparatus for providing an artificial intelligence based VR psychological test service according to an embodiment, a psychological test is performed in the virtual reality, and during the test, a heart rate, a body temperature, and the like are measured, and a psychological state is analyzed, and thus, an accurate test result is provided, and a contactless comfortable and convenient psychological test using VR may be performed.

So far, the present disclosure is described with reference to a preferred embodiment, but it will be apparent to those skilled in the art that various modifications and variations may be made in the present disclosure without departing from the spirit or scope of the disclosure.

[Detailed Description of Main Elements]

| | |
|---|---|
| 100: system for providing a VR | |
| psychological test service | |
| 110: examinee VR terminal | 130: biosignal measurement sensor |
| 150: apparatus for providing VR | |
| psychological test service | |
| 170: database | |
| 210: processor | 230: memory |
| 250: user input/output unit | 270: network input/output unit |
| 310: test contents providing unit | 330: test data collecting unit |
| 350: AI analyzing unit | 370: test result providing unit |
| 390: control unit | |
| 510: test scene | 530: drawing image |

What is claimed is:

1. An apparatus for providing an artificial intelligence based VR psychological test service, comprising:
a test contents providing unit for providing VR contents for a psychological test to an examinee VR terminal and progressing the psychological test through a user interaction with the VR contents;
a test data collecting unit for collecting test data of an examinee; and
an AI analyzing unit for determining a psychological state of the examinee by analyzing the collected test data based on artificial intelligence.

2. The apparatus for providing an artificial intelligence based VR psychological test service of claim 1, wherein the test contents providing unit progresses the psychological test of the examinee in virtual reality by playing the test contents for experiencing a standardized psychological test tool according to a type of the psychological test in the virtual reality through the examinee VR terminal.

3. The apparatus for providing an artificial intelligence based VR psychological test service of claim 2, wherein the test contents providing unit provides a house-tree-person (HTP) drawing test by providing a test scene in front of the examinee through the examinee VR terminal, chasing a movement of a hand of the examinee by a hand glove motion sensor, and displaying a line in a movement direction of the chased hand in the test scene.

4. The apparatus for providing an artificial intelligence based VR psychological test service of claim 1, wherein the test data collecting unit collects biometric data together with the test data by measuring biometric signals of the examinee during the psychological test of the examinee.

5. The apparatus for providing an artificial intelligence based VR psychological test service of claim 4, wherein the test data collecting unit collects the test data in association with at least one biometric data of a heart rate, a body temperature, or a respiratory rate of the examinee.

6. The apparatus for providing an artificial intelligence based VR psychological test service of claim 5, wherein the AI analyzing unit determines a psychological state by analyzing the collected test data through deep learning algorithm and reflecting a test item causing an emotional response through changes of the collected biometric data.

7. The apparatus for providing an artificial intelligence based VR psychological test service of claim 1, further comprising a test result providing unit for generating a test record sheet and a test result sheet of the examinee and providing the test record sheet and the test result sheet to the examinee or a test client.

8. A method for providing an artificial intelligence based VR psychological test service, comprising:
progressing a psychological test of an examinee in virtual reality by playing VR contents for the psychological test;
collecting test data in association with biometric data of the examinee by measuring at least one biometric data of a heart rate and a body temperature during the psychological test of the examinee;
determining a psychological state of the examinee by analyzing the collected test data and the biometric data of the examinee; and
generating and providing a test record sheet and a test result sheet of the examinee.
